(19) United States
(12) Patent Application Publication
(10) Pub. No.: US 2011/0282217 A1
(43) Pub. Date: Nov. 17, 2011

(54) METHOD AND DEVICE FOR DETERMINING DYSFUNCTION OF THE HEART

(75) Inventor: Aws Salim Nashef, Huntington Beach, CA (US)

(73) Assignee: Omega Critical Care Limited, East Kilbride (GB)

(21) Appl. No.: 13/132,359

(22) PCT Filed: Dec. 3, 2009
(86) PCT No.: PCT/GB2009/051639

§ 371 Date: Jul. 25, 2011

Related U.S. Application Data
(60) Provisional application No. 61/200,795, filed on Dec. 3, 2008.

(30) Foreign Application Priority Data
Mar. 9, 2009 (GB) 09040312

(51) Int. Cl. A61B 5/0215; A61B 5/021

(52) U.S. Cl. 600/488; 600/485

(57) ABSTRACT

A method of determining ventricular dysfunction, particularly right ventricular dysfunction and device for determining dysfunction of the heart and determining a cardiac output are discussed. Furthermore a system for determining the pressure(s) and/or the pressure difference gradient between right ventricular diastolic pressure and pulmonary artery diastolic pressure said system comprising a support member (10) adapted to determine the pressure(s) and/or the pressure difference gradient between right ventricular diastolic pressure and pulmonary artery diastolic pressure said support member comprising pressure ports (14, 16) at two spaced apart points wherein the pressure ports provide an input to a monitor wherein the monitor can determine at least one of i) a modulation in the pressure difference gradient between right ventricular diastolic pressure and pulmonary artery diastolic pressure between the first and second later period, and ii) a modulation in the right ventricular diastolic pressure and pulmonary artery diastolic pressure between the first and second later period is discussed.

### FIELD OF THE INVENTION

The present invention relates to a method of determining dysfunction of the heart, more particularly, it relates to a method of determining ventricular dysfunction, particularly right ventricular dysfunction. Furthermore, the present invention relates to a device for determining dysfunction of the heart and determining a cardiac output.

### BACKGROUND OF THE INVENTION

Cardiac output can be determined by using catheters, which have cardiac output determination devices. One method of determining cardiac output involves injecting a cold saline solution into a bloodstream and determining a temperature of the blood, downstream of a site of injection. Alternatively, a portion of blood in the bloodstream can be heated or cooled via a heat transfer device provided in the bloodstream by a catheter and the temperature difference of the blood downstream of the heat transfer device can be determined and compared to normal blood temperature. Additionally, in further alternative methods, a device as discussed in U.S. Pat. No. 5,682,899 or U.S. Pat. No. 5,509,424 can be used wherein a first temperature sensor determines the native temperature of blood, and a second temperature sensor is positioned apart from the first temperature sensor and juxtaposed to a heat transfer device, wherein the heat transfer device promotes efficient radial dissipation of heat without causing an increase in the temperature of the blood.

Whilst the positioning of catheter devices within the heart using pressure sensors able to determine cardiovascular pressures, for example as discussed in WO 01/13789, has been taught, the use of such pressure readings to allow for early diagnosis of dysfunction of the heart, for example to identify complications arising during surgery has not been realised. Further, it was not appreciated that right ventricular pressure and pulmonary artery pressure could be simultaneously measured without requiring a wedge.

### SUMMARY OF THE INVENTION

Right ventricular (RV) pressure monitoring by direct RV catheter is not routinely used because of the possibility of arrhythmias and RV rupture. The present inventor has devised a pulmonary artery catheter which allows safe continuous RV pressure monitoring.

Using this device, the inventor has determined that right ventricular dysfunction can be determined by measuring at least right ventricular pressure and pulmonary artery pressure, particularly by determining the pressure difference gradient between these pressures and that these pressures can be conveniently obtained using a catheter with suitably positioned pressure measuring ports, wherein the catheter may also include means to monitor cardiac output.

According to a first aspect of the present invention there is provided a method of determining right ventricular dysfunction, the method comprising the steps:
determining the right ventricular pressure and pulmonary artery (PA) pressure at a first period in time,
determining the right ventricular pressure and pulmonary artery pressure at a second later period in time,
wherein
i) modulation in the pressure difference gradient between determined right ventricular pressure and the determined pulmonary artery pressure between the first and second later period, or
ii) modulation between the first and second later period in the determined right ventricular pressure and/or the determined pulmonary artery pressure
   is indicative of right ventricular dysfunction.

In embodiments modulation can be a decrease in the pressure difference between the right ventricular pressure and the pulmonary artery pressure. In alternative embodiments, modulation can be an increase in the pressure difference between the right ventricular pressure and pulmonary artery pressure.

In embodiments, the pressures determined can be diastolic pressures. In embodiments the RV and PA pressures can be determined simultaneously and continuously.

In embodiments the determination of the right ventricular pressure and pulmonary artery pressure does not require a wedge in the pulmonary artery to be provided. This is advantageous as should a wedge be provided in a pulmonary artery branch rather than in the pulmonary artery, this can lead to damage. In preferred embodiments the method is provided for use during cardiac surgery.

In an embodiment there is provided a method of determining right ventricular dysfunction, the method comprising the steps of:
determining a pressure difference gradient between right ventricular pressure, particularly right ventricular diastolic pressure, and pulmonary artery pressure, particularly pulmonary artery diastolic pressure, at a first period in time,
determining a pressure difference gradient between right ventricular pressure, particularly right ventricular diastolic pressure, and pulmonary artery pressure, particularly pulmonary artery diastolic pressure, at a second later period in time,
   wherein a modulation in the gradient between the first and second later period is indicative of right ventricular dysfunction.

Determination of the gradient can be determined every 10 seconds or less, preferably every 5 seconds, every 2 seconds, most preferably essentially continuously.

In preferred embodiments the RV and pulmonary artery diastolic pressures are monitored continuously.

In normal cases there should be at least 5 mm Hg pressure differential between the RV and pulmonary artery diastolic pressures. If the difference decreases it indicates that RV is not able to pump blood into the lungs. If this persists without intervention, it can lead to RV failure and result in major deterioration that may require heart assist device.

Right ventricle pressure and pulmonary artery pressure may be determined by respective pressure ports/sensors suitably located on a catheter such that a right ventricle pressure port/sensor can be positioned in the right ventricle at the same time as a pulmonary artery pressure port/sensor can be positioned in the pulmonary artery. The skilled person would understand which catheters, for example catheters as utilised in the measurement of cardiac output, would be of suitable dimension to allow provision of a pressure sensor at these areas in the body.

In embodiments of the method pressures can be determined continuously. By continuously it is meant the reading from the catheter can be taken multiple times a second, for example 2, 5, 7, 10 or more times a second. In alternative embodiments the pressures can be determined for a pre-set or variable period of time. In particular embodiments, the pressures can be displayed, for example on a monitor or the like, continuously or for a pre-set or variable time. In embodiments the pressure readings from a catheter may be shown on a screen wherein the screen updates every second. The pressures can be displayed continuously and substantially in real-time. If the right ventricular pressure and pulmonary artery pressure are determined continuously, the pressure difference gradient can be determined based on the pressures for a pre-set or variable time and/or as a single event.

In embodiments of the method of the invention, when the pressure difference gradient between the first and second later period of time indicates right ventricular end-diastolic pressure greater than 20 mm Hg, it is indicative of right ventricular failure.

In embodiments of the method of the invention, when the right ventricular pressure and pulmonary artery pressure are low, for example when the right ventricular pressure and pulmonary artery pressures are in a range of 0 mmHg to 2 mmHg, this is indicative of hypovolemic shock.

In embodiments, the method can further comprise the step of measuring at least one of right atrial pressure and a wedge pressure.

In a preferred embodiment, elevated right atrial and elevated right ventricle pressure and normal wedge pressure are indicative of right ventricle infarction. An elevated pressure is a pressure value above a normal pressure value, for example at least 150% over normal pressure, at least 175% over normal pressure, at least double normal pressure. For example, normal right atrium (RA) pressure is in a range of 0 mmHg to 5 mm Hg, normal right ventricular systolic pressure (RVSP) is in a range of 15 mmHg to 30 mmHg, right ventricle diastolic pressure (RVEDP) is in a range of 0 mmHg to 8 mmHg and normal wedge pressure is in a range of 2 mmHg to 12 mmHg.

In embodiments when a right arterial pressure is greater than or equal to 10 mmHg, right ventricle diastolic pressure is greater than or equal to 15 mm Hg and wedge pressure is in the range 2-12 mmHg it is indicative of right ventricle infarction.

Particularly, an elevated right atrial pressure which is substantially equal to or above 10 mmHg and an elevated right ventricle diastolic pressure (RVEDPV) which is substantially equal to or above 15 mmHg are indicative of right ventricle infarction.

In embodiments when the difference between pulmonary artery diastolic and right ventricle diastolic (PAD-RVD) is negative, this can be indicative of right ventricle dysfunction.

In embodiments, when the right ventricular pressure is changed from typically expected values, it can be indicative of Tamponade. In particular embodiments, when the right ventricular pressure is increased compared to a normal pressure it is indicative of Tamponade. However, in severe cases, a signal indicative of the right ventricle pressure can be lost.

According to a second aspect of the present invention there is provided a support member adapted to determine the pressure(s) and/or the pressure difference gradient between right ventricular pressure, preferably right ventricular diastolic pressure and pulmonary artery pressure, preferably pulmonary artery diastolic pressure, said support member comprising pressure ports at two spaced apart points, wherein the support member can be a catheter dimensioned for placement within a patient's body cavity having blood flow, for example within a blood vessel and/or the heart. A first pressure port can be located toward the distal end of the support member and be capable of measuring pulmonary artery pressure, and a second pressure port can located at a spaced distance from the first pressure port such that it can measure right ventricular pressure when the first pressure port is measuring pulmonary artery pressure. Preferably diastolic pressures are detected at the first and second pressure ports.

Each of the pressure ports are associated to a lumen within the support member or catheter.

In embodiments the second pressure port can be provided at a spaced distance of about 4.5 cm to 14 cm from the first pressure port. For example, the second pressure port can be located in the range 5.5 cm to 9.5 cm, 5.5 cm to 8.5 cm or in the range 10.5 cm to 13.5 cm from the first pressure port. In embodiments, the first pressure port can be located at a distal end of the catheter, particularly at a distal tip, and the second pressure port can be located at about 4.5 to 14 cm from the distal end of the catheter, preferably 4.5 cm to 9.5 cm, more preferably, 5.5 cm to 8.5, 6.5 to 8.5 cm, 7.5 to 8.5 cm, 8 to 8.5 cm from the distal end or tip of the catheter.

According to a third aspect of the present invention there is a system provided for determining the pressure(s) and/or the pressure difference gradient between right ventricular pressure and pulmonary artery pressure said system comprising a support member adapted to determine the pressure(s) and/or the pressure difference gradient between right ventricular pressure and pulmonary artery pressure said support member comprising pressure ports at two spaced apart points wherein the pressure ports provide an input to a monitor wherein the monitor can determine a modulation in the pressure difference gradient between right ventricular pressure and pulmonary artery pressure between a first and second later period, or modulation in the right ventricular pressure and pulmonary artery pressure between a first and second later period. Suitably diastolic pressures can be determined. In embodiments the monitor can provide an output to signal a modulation in the pressure difference gradient between right ventricular pressure and pulmonary artery pressure between the first and second later period, or modulation in the right ventricular pressure and pulmonary artery pressure between the first and second later period. In embodiments the signal can be provided as a visible signal, for example on a display screen, or as an audible signal.

In embodiments, the support member providing an input to a monitor can be a catheter dimensioned for placement within a patient's body cavity having blood flow, for example within a blood vessel and/or the heart. A first pressure port can be located toward the distal end of the support member and be capable of measuring pulmonary artery pressure, and a second pressure port can located at a spaced distance from the first pressure port such that it can measure right ventricular pressure when the first pressure port is measuring pulmonary artery pressure. Preferably diastolic pressures are detected at the first and second pressure ports. In embodiments the second pressure port can be provided at a spaced distance of about 4.5 cm to 14 cm from the first pressure port. For example, the second pressure port can be located in the range 5.5 cm to 8.5 cm or in the range 10.5 cm to 13.5 cm from the first pressure port. In embodiments, the first pressure port can be located at a distal end of the catheter, particularly at a distal tip, and the second pressure port can be located at about 4.5 to 14 cm from the distal end of the catheter, preferably 4.5 cm to 9.5 cm, more preferably, 5.5 cm to 8.5, 6.5 to 8.5 cm, 7.5 to 8.5 cm, 8 to 8.5 cm from the distal end or tip of the catheter.

Preferably, the modulation in the pressure difference gradient is determined for all pressure difference gradients determined. Alternatively, it can be determined for pre-set or a selected interval of pressure difference gradients.

It is particularly advantageous, if a modulation in the gradient which is indicative of right ventricular dysfunction is automatically detected, for example a modulation of the gradient over a preset value such as a doubling in the change of the pressures between PA and RV can generate an output modulation signal. Alternatively, the modulation can be semi-automatically determined. Furthermore, a corresponding modulation signal can be generated, wherein the modulation signal is indicative of normal heart function and/or right ventricular dysfunction. In embodiments, a signal, for example an audible or visible signal can be provided to a user when modulation in the pressure gradient is detected. For example, a modulation signal can be outputted optically and/or acoustically, for example by a monitor or the like. The modulation signal may be outputted continuously, preferably continuously and substantially in real-time. This is particularly advantageous, as it allows real time monitoring of heart function, for example during intracardiac catheterisation or the like.

In embodiments, it can be advantageous, if a known monitor, e.g. a truCCOMS monitor, is used to determine the modulation in the pressure difference gradient between the right ventricle and pulmonary artery in addition to, for example cardiac output. In embodiments the monitor can provide a signal to a user when the pressure difference gradient between the first and second later period of time indicates right ventricular end-diastolic pressure greater than 20 mm Hg. In embodiments, the monitor can provide a signal to a user when the right ventricular pressure and pulmonary artery pressure are low, for example when the right ventricular pressure and pulmonary artery pressures are in a range of 0 mmHg to 2 mmHg.

In embodiments, the first, second or both pressure port(s) can be selected from any suitable means in the art, for example a diaphragm, fluid-filled lumen, fluid filled piping or the like. Additionally, further pressure port(s) can be located along the length of a body of the catheter, for example to allow measurement of right atrial pressure in addition to right ventricle and pulmonary artery pressure.

In embodiments, the support member can comprise a pressure port at around 25 cm to 35 cm preferably around 30 cm from the distal end capable of determining arterial line pressures to allow central venous pressure (CVP) to be measured. By having both continuously, Systemic vascular resistance (SVR) and Systemic vascular resistance index (SVRI) can be displayed continuously on the monitor screen.

In a further preferred embodiment, the support member, for example a catheter can further comprise a pressure transducer to convert the detected pressure(s) into a corresponding signal. The signal, or a secondary signal calculated from the signal, can be referenced to indicate cardiac dysfunction, particularly right cardiac dysfunction.

In a preferred embodiment the support member can further comprise signal transducers to relay a signal from a first point, for example from a pressure port or a corresponding pressure transducer, along the support member to a proximal end of the support member. The signal transducer can be an existing or additional lumen of the support member, fiber-optics, a conductor or the like, which is capable of relaying the information to the proximal end. Alternatively, in embodiments, a signal transducer can be a transmitter which is capable of transmitting the signal to a corresponding receiver outside a patient's body. Such a receiver can, for example, be located on a portion of the body adjacent to the heart or pulmonary artery and be connected to a computer, monitor and/or similar device.

In particular examples, the pressure can be determined by a diaphragm of the or each pressure port, and fiber-optics of the or each pressure port can be used to detect the pressure(s) and/or to as a transducer to relay the corresponding signal to the proximal end of the catheter.

In embodiments, the pressure difference gradient between the right ventricular diastolic pressure and pulmonary artery diastolic pressure is determined using a system comprising a pressure transducer to convert the measured pressure into a signal, signal transducers to relay at least one of a pressure and a signal from a first point along the catheter to a proximal end of the catheter, a pressure monitor which can calculate the pressure difference gradient over the pulmonary artery valve as the pressure difference between the right ventricle and the pulmonary artery and a catheter comprising a first pressure port located toward the distal end of the catheter said first pressure port capable of measuring pulmonary artery diastolic pressure and a second pressure port located at about 4.5 to 14 cm from the first pressure port, preferably about 4.5 cm to 9.5 cm, more preferably 5.5 cm to 9 cm, 5.5 cm to 8.5 cm 6.5 cm to 8.5 cm, 7.5 cm to 8.5 cm, 8 cm to 8.5 cm, most preferably 8.5 cm said second pressure port capable of measuring right ventricular diastolic pressure.

As will be appreciated finding that right ventricular dysfunction can be determined based on right ventricular pressure and pulmonary artery pressure, particularly a pressure difference gradient between these pressures or more particularly based on a gradient between two of these pressure difference gradients determined at different times, provides for an improved multi function support member for determining ventricular dysfunction, particularly right ventricular dysfunction, and for determining a blood flow in a pulmonary artery, wherein such an improved support member provides for an improved functionality and is more safely and conveniently used. The support member may be a suitably sized catheter.

Accordingly, a further aspect of the present invention provides a catheter for use in the methods of determining right ventricular dysfunction, as hereinbefore and hereinafter described, and blood flow in the pulmonary artery, wherein the catheter comprises

a catheter body with a distal end and a proximal end, a first pressure port located toward the distal end of the catheter, wherein the first pressure port is capable of measuring pulmonary artery pressure, particularly pulmonary artery diastolic pressure, and a second pressure port located at about 4.5 cm to 14 cm from the distal end, preferably about 4.5 cm to 9.5 cm, preferably 4.5 cm to 9 cm, more preferably 5.5 cm to 8.5 cm, 6.5 cm to 8.5 cm, 7.5 cm to 8.5 cm, 8 cm to 8.5 cm, most preferably 8.5 cm. wherein the second pressure port is capable of measuring right ventricle pressure, particularly right ventricle diastolic pressure, the catheter further comprising a first temperature sensor, a heat transfer device, and a second temperature sensor wherein the heat transfer device is interposed between the pressure ports, the first temperature sensor is juxtaposed to the heat transfer device and the second temperature sensor is capable of measuring the native temperature of blood. In preferred embodiments, the heat transfer device is located in the range of about 2 to 3.5 cm from the distal tip of the catheter, more preferably about 2 to 3 cm from the distal tip of the catheter, most preferably at about 2.5 cm from the distal tip of the catheter. In embodiments wherein the heat transfer device is located at between 2 to 3.5 cm from the distal tip of the catheter, more preferably 2 to 3 cm from the distal tip of the catheter, most preferably 2.5 cm from the distal tip of the catheter the second pressure port can be about 6-8.5 cm from the distal tip of the catheter.

In embodiments, the heat transfer device can be positioned adjacent to the first temperature sensor and spaced apart from the second temperature sensor. The heat transfer device can comprise a heating device positioned between a heat conducting layer and an insulating layer. The insulating layer forms an outer layer of the heating device, such that the insulating layer is in contact with blood and the heating device is in thermal communication with the blood when the heat transfer device is positioned within the pulmonary artery. The heat conducting layer is positioned to the inside of the heating device and the first sensor so as to be in thermal contact with the first sensor, and the heating device is capable of increasing the temperature of the heat transfer device to a second temperature above the native temperature of the blood, as determined by the second sensor.

In embodiments, the support member, for example a catheter, can comprise a heat transfer device to measure cardiac output such as those discussed in U.S. Pat. No. 5,682,899, U.S. Pat. No. 5,509,424 or WO01/1380, which are hereby incorporated by reference. For example, the heat transfer device can include a temperature sensor juxtaposed to a heat transfer device, wherein the heat transfer device promotes efficient radial dissipation of heat without causing a significant increase in the temperature of the blood. In particular embodiments the heat transfer device can maintain a differential of around 2 degrees Celsius above native blood temperature. In embodiments, native blood temperature is measured by a temperature sensor, for example a thermistor which is spaced apart from the heat transfer device whilst another temperature sensor is provided juxtaposed to the heat transfer device to measure the temperature of the heat transfer device. As blood flows across the heat transfer device, the heat transfer device is cooled, requiring further power to be supplied to the heat transfer device to maintain the 2 degrees Celsius differential. As will be appreciated, if cardiac output increases more power is required, if it decreases, less power is required. The power required is thus proportional to blood flow and allows measurement of blood flow.

In embodiments, the heat transfer device can be interposed between the first pressure port and the second pressure port. In particular embodiments, the mid point of the heat transfer device can be located at about 2 to 7.5 cm, preferably 2 to 6 cm, 2.5 cm to 6 cm proximal, preferably 4.5 cm to 6 cm proximal of the first pressure port. For example, if the mid point of the heat transfer device is located at 7.5 cm from the distal end of the catheter, the first pressure port, which in use determines pulmonary artery pressure, can be located substantially at the distal end of the catheter and the second pressure port, which in use determines right ventricle pressure, can be located in the range 10.5 cm to 13.5 cm from the distal end of the catheter. In embodiments wherein the mid point of the heat transfer device is located at between 2 to 3.5 cm from the distal end of the catheter, more preferably 2 to 3 cm from the distal end of the catheter, most preferably 2.5 cm from the distal end of the catheter, the first pressure port can be located substantially at the distal end of the catheter, and the second pressure port can be located in the range 5.5 cm to 8.5 cm from the distal end of the catheter.

The spacing of the heat transfer device relative to the pressure ports is advantageous as it allows the heat transfer device to be correctly positioned without requiring a wedge to be performed and thus minimises the risk of damage, whilst also allowing the pressure gradient between the right ventricle and pulmonary artery to be determined.

Preferably, the pressure(s) can be determined by fiber-optics, which advantageously results in a compact catheter that can more conveniently be used.

As will be appreciated, it is particularly convenient and cost efficient if a known monitoring system for monitoring cardiac flow, such as a truCCOM monitor or the like, is adapted to display and/or determine the pressure(s), pressure difference gradient(s) and/or gradient between the pressure difference gradients determined at a first and second period in time.

In embodiments of a support member of the invention, a catheter can comprise a third pressure port capable of determining central venous pressure (CVP) which can be located spaced apart from the distal end of the catheter, particularly at 25 cm to 35 cm, preferably 30 cm from the distal end. It is particularly advantageous if a port already present in the catheter is adapted as the third pressure port, for example the port which is normally used for injection of saline for thermodilution measurements or the like.

Embodiments of the invention will now be described by way of example only, with reference to the accompanying figures in which;

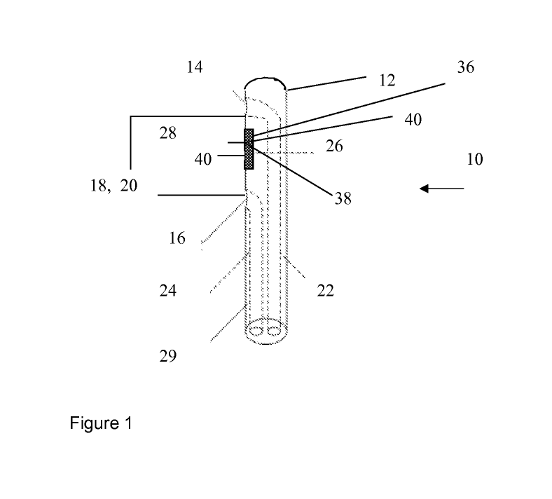

FIG. 1 shows a schematic representation of an embodiment of a catheter according to the present invention,

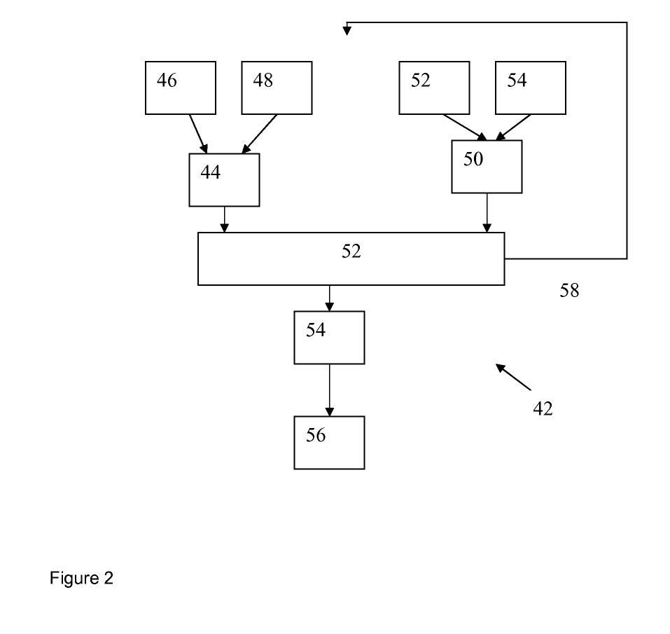

FIG. 2 shows a schematic representation of a method according to the present invention,

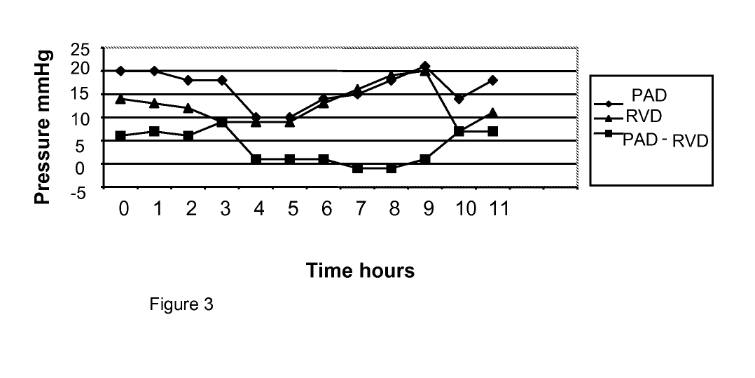

FIG. 3 illustrates a line graph showing the different pressures with respect to time determined from a subject using a catheter of the present invention from the arrival in cardiac surgery intensive care unit at 3 hours in stable condition, becoming haemodynamically unstable between 4 and 9.5 hours, opening the subject's chest and removal of a clot,

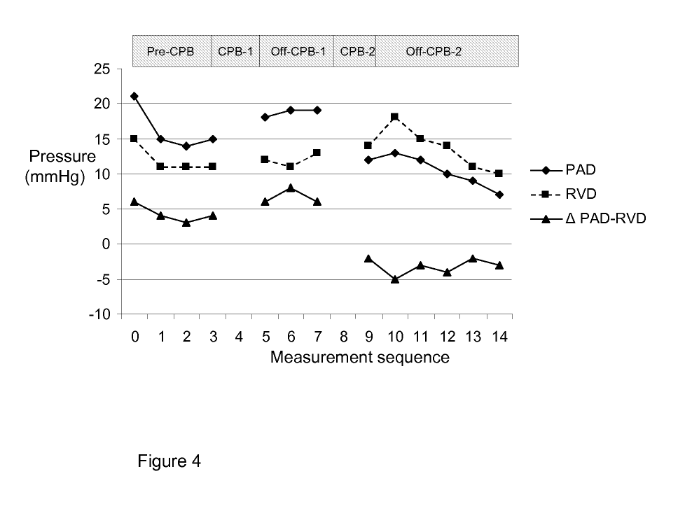

FIG. 4 shows a graph of pulmonary artery diastolic (PAD), right ventricle diastolic (RVD), difference between pulmonary artery diastolic and right ventricle diastolic (ΔPAD-RVD) over a 6 hour intraoperative period, with first and second cessation of cardiopulmonary bypass (CPB) at 2 and 4 hours post induction respectively, and

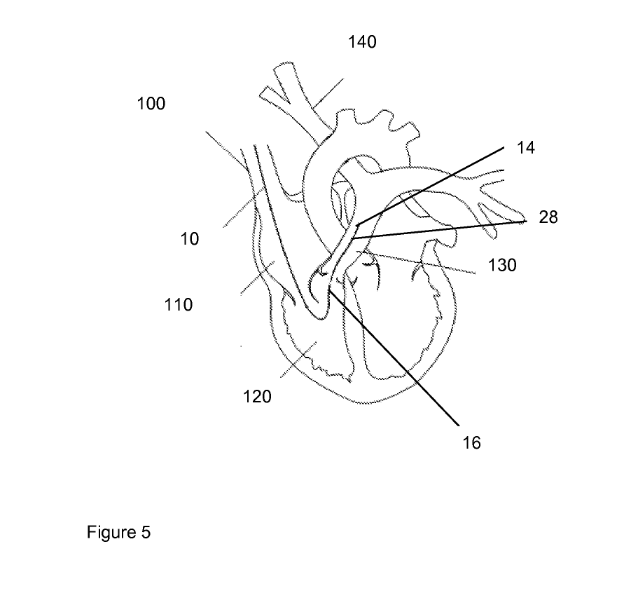

FIG. 5 shows an illustration of a support member when positioned in the heart, with the superior vena cava 100, Right Atrium 110, Right Ventricle 120, Pulmonary Artery 130, and Right Pulmonary Branch 140 being indicated with a first pressure port being located in the pulmonary artery and a second pressure port being located in the right ventricle to allow measurement of the pressure gradient between the pulmonary artery and right ventricle wherein the tip of the support member is not advanced towards the branches of the pulmonary artery and positioning does not require a wedge.

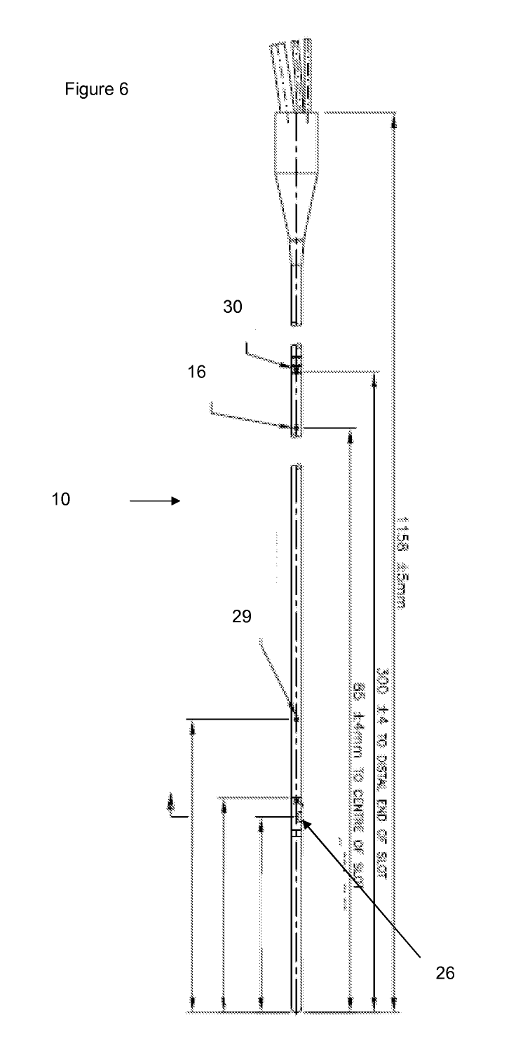

FIG. 6 shows an embodiment of a support member of the present invention.

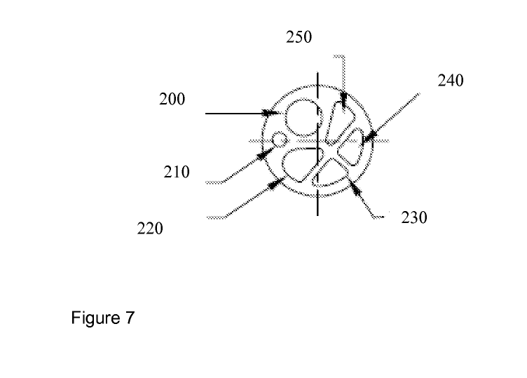

FIG. 7 illustrates the internal lumen structure of an embodiment of the support member of the present invention.

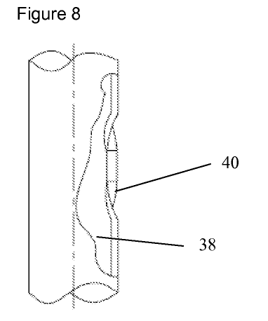

FIG. 8 illustrates a schematic portion of the support member including the heat transfer device with the insulating layer 38 and heat conducting layer 40 being identified.

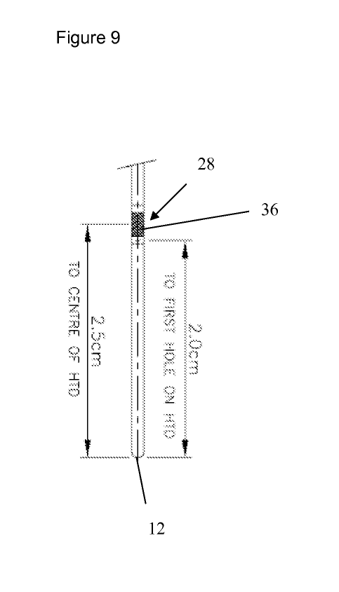

FIG. 9 illustrates the location of the heat transfer device (HTD) in an embodiment of a support member of the invention with the HTD beginning at 2 cm from the tip and the midpoint of the HTD being at 2.5 cm from the tip.

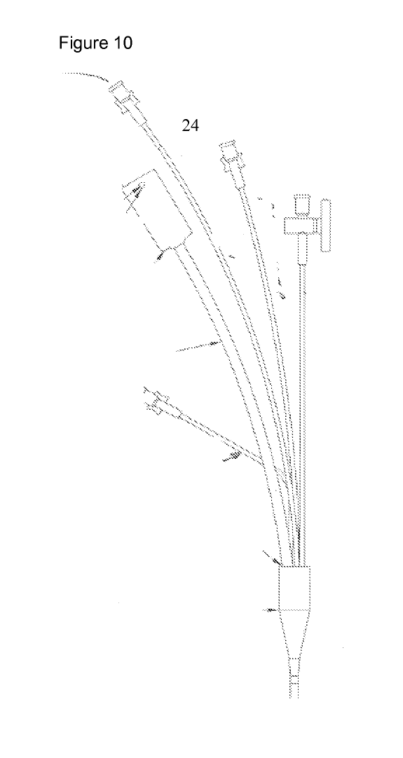

FIG. 10 illustrates an embodiment of a proximal end of the support member.

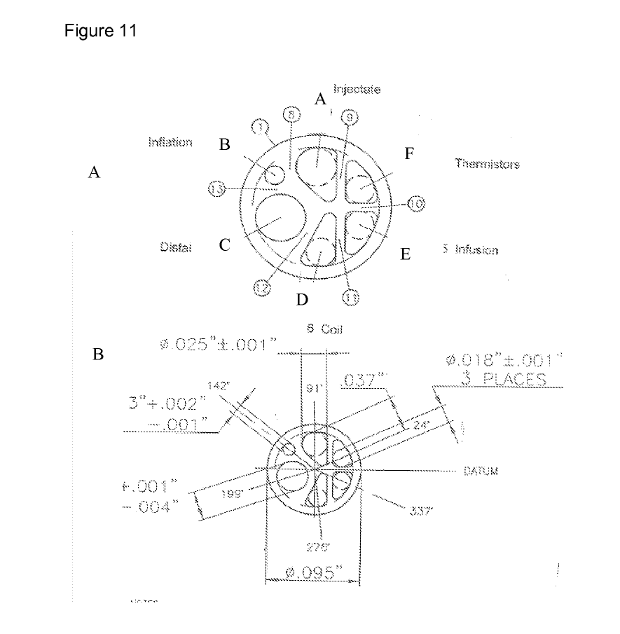

FIGS. 11 A and B illustrate the internal configuration of a 6 lumen support member wherein lumen A 220 is for proximal injectate slot or central venous pressure port, lumen B 210 is for inflation, lumen C 200 is for a first pressure port at the distal tip, lumen D 250 is for the coil, lumen E 240 is for the second pressure port for lumen F 230 is for thermistors. As noted in FIG. 11B lumen A is around 0.025 inches, lumen B is around 0.024 inches, lumen C is around 0.024 inches, lumen D is around 0.024 inches, lumen E is around 0.0024 inches, and lumen F is around 0.024 inches.

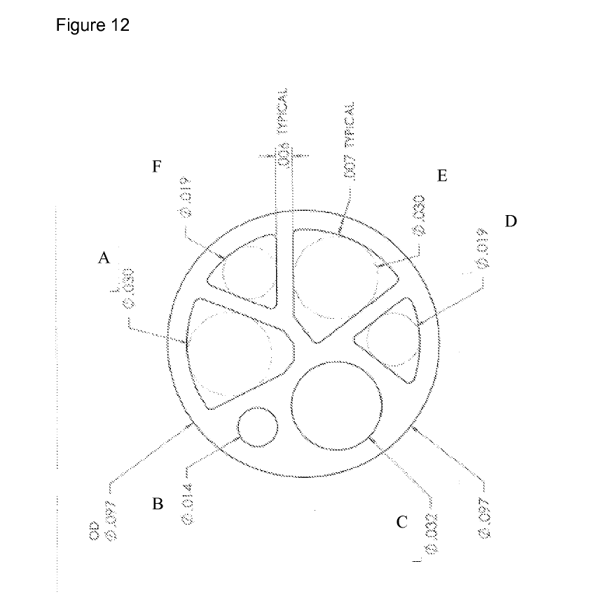

FIG. 12 shows an alternative lumen with modified dimensions to try and improve pressure readings wherein lumen A is around 0.034 inches, lumen B is around 0.0014 inches, lumen C is around 0.0032 inches, lumen D is around 0.019 inches, lumen E is around 0.030 inches and lumen F is around 0.019 inches.

FIG. 1 shows a schematic representation of an embodiment of a catheter 10 according to the present invention, wherein the catheter 10 has a distal end, which comprises a catheter tip 12. A first pressure port 14 is located adjacent to the distal end of the catheter 10 and a second pressure port 16 is located spaced apart from the first pressure port 14 at a proximal end of the catheter 10.

As shown in FIG. 1, the second pressure port can be located at 5.5 cm to 8.5 cm from the distal end of the catheter with the first pressure port 14.

The first pressure port can be located at the catheter tip 12.

In alternative embodiments not illustrated, the second pressure port can be located at 10.5 cm to 13.5 cm from the distal end of the catheter.

The pressure ports 14, 16 are shown as diaphragms; however, a fluid filled lumen or piping or another means capable of pressure determination can be used.

A respective first and second pressure transducer 18, 20 capable of converting the pressure(s) into a signal are in communication with the first and second pressure ports 14, 16 and are connected with a respective first and second signal transducer 22, 24 capable to relay the signal along the catheter 10 to the proximal end of the catheter 10.

Alternatively, a single pressure transducer can be in communication with both pressure ports 14, 16 to convert the pressures and can be connected with one or two signal transducers.

As will be appreciated, the signal transducer(s) 22, 24 can be an existing or additional lumen of the catheter 10, fiber-optics, conductor or similar.

A first temperature sensor 26 is located adjacent a heat transfer device 28 on the catheter 10, and a second temperature sensor 29 capable of detecting a native blood temperature is located spaced apart from the first temperature sensor 26 on the catheter 10. The heat transfer device is advantageously located about 2.5 cm from the distal end of the catheter. As will be appreciated, the second temperature sensor 29 can be located upstream or downstream of the second pressure port 16.

The first and second temperature sensor 26, 29 and the heat transfer device 28 can be connected with a corresponding signal transducer and/or power supply, represented by conductors.

A heating device 36 of the heat transfer device 28 is positioned between an insulating layer 38, which is an outer layer of the heating device 28, and a heat conducting layer 40. The heat conducting layer 40 is positioned to an inside of the heating device 28 and to the first temperature sensor 26, such that the first temperature sensor 26 is capable of determining a temperature of the heating device 36.

Alternatively, the first temperature sensor 26 can be capable of determining a resistance of a heating wire comprised by the heating device 36, such that, if a constant voltage is applied to the heating wire, a change in resistance can be detected, which is indicative of a blood flow.

FIG. 2 shows a schematic representation of a method 42 according to the present invention, comprising the steps of determining a pressure difference gradient—first pressure difference gradient 44—between right ventricular diastolic pressure 46 and pulmonary artery diastolic pressure 48 at a first period in time, and determining a pressure difference gradient—second pressure difference gradient 50—between right ventricular diastolic pressure 52 and pulmonary artery diastolic pressure 54 at a second, later, period in time.

First and second pressure difference gradients 44, 50 are compared 52 in order to detect a modulation 54 of the pressure difference gradients 44, 50, which is indicative of right ventricular dysfunction 56. If the step of comparing 52 the pressure difference gradients 44, 50 does not indicate the modulation 54, the method 42 can be repeated 58.

### EXAMPLE 1

### The Use of Continuous Monitoring of Right Ventricular and Pulmonary Artery Diastolic Pressures in Cardiac Surgery

A dedicated catheter to continuously monitor pressure in the right ventricle (RV) during cardiac surgery is not routinely used because of risks of arrhythmias or RV perforation. The study determined how right ventricular diastolic pressures change during cardiac surgery involving cardiopulmonary bypass.

### Method

Seven patients participated in a prospective ethically approved study using a catheter of the present invention to determine how RV pressures change during various stages of cardiac surgery involving cardiopulmonary bypass. In particular the study was to determine how the PA diastolic, RV diastolic and delta (A) PA diastolic—RV diastolic pressures vary at 4 perioperative set times: (1) prior to anaesthesia induction and (2) (3) and (4) at 30 minutes after induction, protamine administration and arrival in cardiac surgical intensive care unit (CSICU) respectively.

### Results

The mean values along with standard deviation are presented in Table 1. All values were compared with baseline using Friedman's and Dunn's multiple comparisons.

TABLE 1
Mean values for pulmonary artery diastolic (PAD), right
ventricular diastolic (RVD) pressures and difference
between them (Δ PAD − RVD).

| | | | 30 min | 30 min after |
|---|---|---|---|---|
| | | 30 min post | post | arrival to |
| | Baseline | anaesthesia | protamine | CSICU |
| PAD mmHg | 16.8 ± 2.5 | 15.4 ± 2.4 | 16.3 ± 3.9 | 11.7 ± 3.14 |
| (mean ± SD) | | | | |
| RVD mmHg | 12.1 ± 1.9 | 11 ± 2 | 11.3 ± 3.5 | 8.7 ± 1.6* |
| (mean ± SD) | | | | |
| Δ PAD − RVD | 4.7 ± 1.2 | 4.4 ± 1.3 | 5 ± 3.5 | 3 ± 3 |
| mmHg | | | | |
| (mean ± SD) | | | | |

RVD 30 minutes after arrival in CSICU was lower than baseline (*P<0.05).

This study demonstrates normal values for ΔPAD-RVD pressures in routine cardiac surgery where PAD is greater than RVD throughout the study period.

### EXAMPLE 2

### Use of Continuous Right Ventricular Diastolic Pressure Monitoring in Detecting Cardiac Tamponade in the Cardiac Surgical Intensive Care Unit

Presence of postoperative pericardial tamponade at cardiac surgery is suggested by a combination of reduced systemic and elevated central venous pressures with normal or low pulmonary artery diastolic pressure. This diagnosis can sometimes be confirmed by transoesophageal echocardiography. Continuous right ventricular diastolic pressure monitoring using a catheter of the present invention was performed and determination of right ventricular pressure was used in the diagnosis and monitoring of treatment for pericardial tamponade following cardiac surgery. In the embodiment of the catheter used the pulmonary artery catheter was provided with lumens for simultaneous and continuous monitoring of central venous, pulmonary artery (PA) and right ventricle (RV) pressures.

### Case Report

A 73-year old male underwent coronary artery bypass grafting (CABG) and aortic valve replacement (AVR). His medical history revealed moderate aortic stenosis with good left ventricular function, triple vessel disease, hypertension and non-insulin dependent diabetes mellitus. In the study using a catheter of the present invention the change in RV pressure during cardiac surgery involving cardiopulmonary bypass was determined. Pulmonary artery (PA) diastolic, right ventricle (RV) diastolic and delta PA diastolic-RV diastolic (ΔPAD-RVD) pressures intra and post operatively were determined. Values for ΔPAD-RVD at baseline, 30 minutes post induction, 30 minutes after protamine administration and 30 minutes after intensive care unit (ICU) admission were +6, +7, +6, and +9 mmHg respectively. However, shortly after this the patient became haemodynamically unstable needing fluids and inotropic support for 6 hours. During this time right ventricle diastolic (RVD) pressure continued to increase exceeding pulmonary artery diastolic (PAD) pressure and ΔPAD-RVD became negative. The chest was reopened in the intensive care unit. A blood clot was identified posterior to the right atrium. Within seconds of clot removal, right ventricle diastolic (RVD) pressure dropped to 7 from 20 mmHg and ΔPAD-RVD pressure returned to baseline of 7 mmHg. Systemic pressures normalised. Subsequently, the patient made a complete recovery.

As illustrated in FIG. 3, the line graph shows the different pressures with respect to time. The patient arrived in cardiac surgery intensive care unit (CSICU) at 3 hours in stable condition. He became haemodynamically unstable between 4 and 9.5 hours when his chest was reopened and clot was removed. The catheter of the present invention enabled diagnosis of acute pericardial tamponade of the subject.

### EXAMPLE 3

### Diagnosis of Right Ventricular Failure After Aortic Valve Replacement Using the Quadlumen Trucath Pulmonary Artery Catheter: a Case Report

Using a catheter of the present invention, continuous monitoring of RV diastolic pressures in a patient detected the presence of RV failure and its response to therapy following cardiopulmonary bypass (CPB).

A 53 year old man underwent aortic valve replacement (AVR) for moderate aortic stenosis and regurgitation. His ejection fraction was normal, but he had increased ventricular volumes and a small occluded right coronary artery. After cessation of CPB for insertion of a St. Jude mechanical valve, the patient was haemodynamically stable and the difference between pulmonary artery diastolic (PAD) and right ventricular diastolic (RVD) pressures (ΔPAD-RVD) was +4 mmHg. However, a small paravalvular leak necessitated repeat cardiopulmonary bypass (CPB) for repair. After the second cessation of cardiopulmonary bypass (CPB), there was evidence of severe right ventricular dysfunction and ΔPAD-RVD was −2 mmHg. For three days the patient remained in severe right ventricular (RV) dysfunction with very high lactates refractory to several treatment modalities including inotropes (milrinone, dopexamine, adrenaline and noradrenaline), nitric oxide and reopening of chest (day 1), intra-aortic balloon pump (day 2). Later on day 2, persisting high lactates and metabolic acidosis prompted a laparotomy to exclude ischaemic bowel. Throughout this time ΔPAD-RVD remained negative. Eventually RV function improvement was accompanied by ΔPAD-RVD values returning to baseline of greater than +5 mm Hg. The patient made a full recovery.

As shown in FIG. 4 a graph of pulmonary artery diastolic (PAD), right ventricle diastolic (RVD) and ΔPAD-RVD over a 6 hour intraoperative period indicated first and second cessations of CPB were at 2 and 4 hours post induction respectively.

The continuous monitoring of ΔPAD-RVD appears to be useful in the early diagnosis and management of RV failure.

### EXAMPLE 4

### Embodiment of Catheter

FIG. 6 illustrates an embodiment of the device of the present invention wherein the catheter 10 has a tip 12 with a first pressure port 14 located at said tip. In use the first pressure port can measure pulmonary artery pressure. A second pressure port with a mid point located at 8.5 cm+/−0.4 cm from the tip 12 is also provided on the catheter. In use the second pressure port can measure right ventricle pressure. A third pressure port (30) located around 30 cm+/−0.4 cm from the tip 12 can, in use, be used to measure central venous pressure. A heat transfer device 28, as described by U.S. Pat. No. 5,682,899; U.S. Pat. No. 5,509,424 or WO 01/1380 is provided at between 2.2 cm to 2.5 cm from the distal tip (midpoint around 2.5 cm+/−0.2 cm). A thermistor 26 to determine the temperature of the heat transfer device is provided with a second thermistor 29 located around 6.5 cm from the distal tip 12.

The catheter as illustrated by FIG. 6 can have an internal configuration of lumens as illustrated in FIG. 7. As illustrated in FIG. 7 an embodiment of a catheter 10 can be provided with 6 lumens; a distal lumen 200, an inflation lumen 210, a central venous pressure/proximal injectate lumen 220, thermistor lumen 230, RV pressure lumen 240, and coil lumen 250.

Although not shown, it will be appreciated that in embodiments of the device which do not include a heat transfer device to measure cardiac output, a thermistor lumen and coil lumen would not be required and thus a four lumen catheter could be formed.

In the embodiment illustrated, the six lumen catheter body is manufactured from a yellow PVC compound which is typically supplied in lengths of 55 inches (55″). FIG. 11 shows the dimensions of the outer walls (0.008 inches) and internal walls (0.005 inches), and individual sizes of the 6 lumens.

The present inventors consider that an improved pressure reading may be obtained if a larger diameter of lumen is provided.

In view of this in embodiments the RV pressure lumen 240 may be provided with dimensions of 0.030″ (FIG. 12)

Various modifications may be made to the invention herein described without departing from the scope thereof.

1. A method of determining ventricular dysfunction, the method comprising the steps:
determining the right ventricular pressure and pulmonary artery pressure at a first period in time, determining the right ventricular pressure and pulmonary artery pressure at a second later period in time, wherein i) modulation in the pressure difference gradient between right ventricular pressure and pulmonary artery pressure determined at the first and second later period, or ii) modulation in the right ventricular pressure and pulmonary artery pressure determined at the first and second later period is indicative of ventricular dysfunction.

2. The method according to claim 1 wherein the pressure difference gradient between right ventricular pressure and pulmonary artery pressure is determined using a catheter comprising a first pressure port located toward the distal end of the catheter said first pressure port capable of measuring pulmonary artery pressure and a second pressure port located at about 4.5 cm to 14 cm from the first pressure port, said second pressure port capable of measuring right ventricular pressure.

3. The method according to claim 1 wherein the catheter comprises a first pressure port located at a distal tip of the catheter, said first pressure port capable of measuring pulmonary artery diastolic pressure and a second pressure port located at about 4.5 to 14 cm from the distal tip of the catheter said second pressure port capable of measuring right ventricular diastolic pressure.

4. The method according to claim 2 wherein a pressure port is selected from a diaphragm, fluid-filled lumen or fluid filled piping.

5. The method according to claim 2 wherein the catheter further comprises a pressure transducer to convert the measured pressure(s) into a signal.

6. The method according to claim 2 wherein the catheter further comprises signal transducers to relay a signal from a first point along the catheter to a proximal end of the catheter.

7. The method according to claim 2 wherein pressure(s) is determined by having a diaphragm on a pressure port and pressure(s) is measured by fiberoptics.

8. The method according to claim 1 wherein when the gradient between the first period and second later period indicates right ventricular end-diastolic pressure greater than 20 mm Hg, it is indicative of right ventricle failure.

9. The method according to claim 1 wherein when the right ventricular pressure and pulmonary artery pressures are less than 2 mmHg it is indicative of hypovolemic shock.

10. The method according to claim 1 further comprising measuring at least one of right atrial pressure and wedge pressure.

11. The method according to claim 10 wherein when a right atrial pressure is greater than or equal to 10 mmHg, right ventricle diastolic pressure is greater than or equal to 15 mm Hg and wedge pressure is in the range 2-12 mmHg it is indicative of right ventricle infarction.

12. The method according to claim 1 wherein when the right ventricular pressure is modulated it is indicative of Tamponade.

13. The method according to claim 1 wherein the pressure difference gradient between the right ventricular pressure and pulmonary artery pressure is determined using a system comprising a pressure transducer to convert the measured pressure(s) into a signal, signal transducers to relay at least one of a pressure and a signal from a first point along the catheter to a proximal end of the catheter, a pressure monitor which can calculate the pressure difference gradient over the pulmonary artery valve as the pressure difference between the right ventricle and the pulmonary artery and a catheter comprising a first pressure port located toward the distal end of the catheter said first pressure port capable of measuring pulmonary artery pressure and a second pressure port located at about 4.5 to 14 cm from the first pressure port, said second pressure port capable of measuring right ventricular pressure.

14. The method according to claim 1 wherein a wedge is not required to locate the catheter such that the first pressure port is suitably located to determine pulmonary artery pressure and the second pressure port is suitably located to determine right ventricle pressure.

15. The method according to claim 1 wherein the second pressure port is located at between 4.5 to 9.5 cm, preferably 5.5 to 8.5 cm, most preferably 8.5 cm from the first pressure port at the distal tip of the catheter.

16. The method according to claim 1 wherein the determined pulmonary artery pressure and right ventricle pressure is diastolic pressure.

17. A catheter for use in the method of claim 1 for determining ventricular dysfunction and blood flow in the pulmonary artery, said catheter comprising:
a catheter body having a distal end and a proximal end;
a first pressure port located toward the distal end of the catheter said first pressure port capable of measuring pulmonary artery diastolic pressure;
a second pressure port located at about 4.5 to 14 cm from the distal end said second pressure port capable of measuring right ventricle pressure;
a first temperature sensor;
a second temperature sensor capable of measuring the native temperature of blood;
a heat transfer device located in the range 2 to 3.5 cm from the distal end of the catheter wherein the heat transfer device is adjacent to the first temperature sensor and spaced apart from the second temperature sensor, said heat transfer device comprising a heating device positioned between a heat conducting layer and an insulating layer, the insulating layer forming an outer layer of the heating device such that the insulating layer is in contact with blood and the heating device is in thermal communication with the blood when the heat transfer device is positioned within the pulmonary artery, the heat conducting layer being positioned to the inside of the heating device and the first temperature sensor so as to be in thermal contact with the first temperature sensor and the heating device capable of increasing the temperature of the heat transfer device to a second temperature above said first temperature.

18. A catheter as claimed in claim 17 wherein the heat transfer device is located at 2.5 cm from the distal end of the catheter.

19. A support member that determines the pressure(s) and/or the pressure difference gradient between right ventricular diastolic pressure and pulmonary artery diastolic pressure, said support member comprising:
pressure ports at two spaced apart points;
a first pressure port located toward the distal end of the support member and which is capable of measuring pulmonary artery pressure; and
a second pressure port located at a spaced distance from the first pressure port such that it can measure right ventricular pressure when the first pressure port is measuring pulmonary artery pressure.

20. A support member according to claim 19 wherein the second pressure port is provided at a spaced distance of about 4.5 cm to 14 cm from the first pressure port.

21. A support member according to claim 20 wherein the second pressure port is provided at a spaced distance of about 4.5 cm to 9.5 cm from the first pressure port, more preferably 5.5 cm to 8.5 cm from the first pressure port.

22. A support member according to claim 19 wherein the first pressure port is located at the distal tip of the support member and the second pressure port is provided at a spaced distance of about 4.5 to 14 cm from the distal tip of the support member and the first pressure port is provided at the distal tip of the support member.

23. A support member according to claim 19 wherein the first pressure port is located at the distal tip of the support member and the second pressure port is provided at a spaced distance of about 4.5 cm to 9.5 cm, more preferably 5.5 cm to 8.5 cm from the first pressure port.

24. A catheter according to claim 17 further comprising a pressure port located between 25 cm to 35 cm, preferably about 30 cm, from the distal tip of the catheter or support member.

25. A catheter according to claim 17 further comprising a right ventricle pressure lumen of diameter around 0.030 inches.

26. A system for determining the pressure(s) and/or the pressure difference gradient between right ventricular diastolic pressure and pulmonary artery diastolic pressure, said system comprising:
a support member that determines the pressure(s) and/or the pressure difference gradient between right ventricular diastolic pressure and pulmonary artery diastolic pressure;
wherein said support member comprises pressure ports at two spaced apart points;
wherein the pressure ports provide an input to a monitor;
wherein the monitor determines at least one of:
i) a modulation in the pressure difference gradient between right ventricular diastolic pressure and pulmonary artery diastolic pressure between the first and second later period; and
ii) a modulation in the right ventricular diastolic pressure and pulmonary artery diastolic pressure between the first and second later period.
